(19) United States
(12) Patent Application Publication
(10) Pub. No.: US 2011/0218627 A1
(43) Pub. Date: Sep. 8, 2011

(54) SYSTEM AND METHOD FOR REPLACING AT LEAST A PORTION OF A VERTEBRAL BODY

(75) Inventors: Y. Raja Rampersaud, Toronto (CA); Anthony J. Melkent, Memphis, TN (US); Jinia Shembekar, New York, NY (US)

(73) Assignee: Warsaw Orthopedic, Inc., Warsaw, IN (US)

(21) Appl. No.: 12/716,857

(22) Filed: Mar. 3, 2010

(51) Int. Cl. A61F 2/44; A61B 17/00

(52) U.S. Cl. 623/17.11; 606/84

(57) ABSTRACT

Embodiments of the invention include systems and methods for replacing at least a part of a vertebral body. Systems may include end components for interfacing with adjacent vertebrae and a central component for placement between end components. Instruments for placing system components and preparing anatomical structures to receive system embodiments may also be included.

### FIELD OF THE INVENTION

The present invention relates generally to the field of stabilizing a spinal structure, and in some embodiments more particularly relates to an implant and method for replacing all or a portion of a vertebral body.

### BACKGROUND

It is sometimes necessary to remove one or more vertebrae, or a portion of the vertebrae, from the human spine in response to various pathologies. For example, one or more of the vertebrae may become damaged as a result of tumor growth, or may become damaged by a traumatic or other event. Removal, or excision, of a vertebra may be referred to as a vertebrectomy. Excision of a generally anterior portion, or vertebral body, of a vertebra may be referred to as a corpectomy. An implant is usually placed between the remaining vertebrae to provide structural support for the spine as a part of a corpectomy or vertebrectomy. This may generally be referred to as vertebral body replacement. In some cases, the implant inserted between the vertebrae is designed to facilitate fusion between remaining vertebrae. In other cases, especially when treating tumors, the ultimate goal of the procedure is spinal stability, regardless of fusion. A successful procedure may decrease pain, preserve or enhance neurological function and allow a patient greater mobility without an external orthosis.

A smaller incision and lesser disruption to surrounding tissues may be particularly helpful to improve outcomes and reduce complications and recovery times. However, small incisions and reduced disruption to surrounding tissues may be difficult with relatively large devices typically associated with complete or partial vertebral body replacements. One solution to this problem is to provide vertebral body replacement devices in multiple pieces that are assembled in situ. Although various vertebral replacement devices of this type are available, improved devices and methods that are capable of avoiding critical anatomical structures, such as neural and vascular structures, and provide adequate structural support, are needed.

### SUMMARY

An embodiment of the invention is a vertebral body replacement system. The vertebral body replacement system may include a spreader instrument with a first tip and a second tip. The spreader instrument may be operable to move the first tip and the second tip farther apart from one another and closer to one another. The system may also include a first end component having a height, a width, and a depth, wherein the depth is greater than the width, and wherein the first end component is configured to couple with the first tip, and wherein an extent of the height of the first end component is configured to be placed against a first adjacent vertebral body. The system may also include a second end component having a height, a width, and a depth, wherein the depth is greater than the width, and wherein the second end component is configured to couple with the second tip, and wherein an extent of the height of the second end component is configured to be placed against a second adjacent vertebral body. At least one central component having a height, a width, and a depth, wherein the depth is greater than the width, and wherein the at least one central component is configured to be placed between the first end component and the second end component may be included in the system.

Another embodiment of the invention is a method of stabilizing a spine from which a vertebral body will be at least partially removed. The method may include placing a first end component against a first adjacent vertebral body, including moving the first end component through a space between the first adjacent vertebral body and a nerve root extending from a spinal cord, and placing a second end component against a second adjacent vertebral body opposite from the first end component. The method may also include inserting a first central component between the first end component and the second end component, including moving the first central component through a space between the second end component and the nerve root extending from the spinal cord.

Still another embodiment of the invention is a method of performing a vertebral body replacement. The method may include implanting a first end component that includes a first track against a first adjacent vertebral body. The first vertebral body is on a first side of the vertebral body to be replaced, and the first track is oriented toward the vertebral body to be replaced. The method may also include implanting a second end component that includes a second track against a second adjacent vertebral body. The second adjacent vertebral body is on a second side of the vertebral body to be replaced that is opposite from the first side, and the second track is oriented toward the vertebral body to be replaced. The method of some embodiments also includes moving a first osteotome in the first track and the second track to cut tissue between the first track and the second track.

### BRIEF DESCRIPTION OF THE DRAWINGS

FIG. 1 is a perspective view of portions of an embodiment of a vertebral body replacement system in use between vertebrae.

FIG. 2 is a perspective view from a generally anterior direction of portions of the system of FIG. 1.

FIG. 3 is a perspective view of portions of the system of FIG. 1.

FIG. 4A is a plan view of an end component of the system of FIG. 1.

FIG. 4B is an elevation view of a side of the component of FIG. 4A.

FIG. 4C is an elevation view of a side of the component of FIG. 4A.

FIG. 5 is a perspective view of components of portions of the system of FIG. 1.

FIG. 6 is a perspective view of some of the components of FIG. 5 being assembled.

FIG. 7 is a perspective view of some of the components of FIG. 5 being assembled.

FIG. 8 is a perspective view of portions of an embodiment of a vertebral body replacement system in use between vertebrae.

FIG. 9 is a perspective view of portions of the system of FIG. 8 in use on a vertebra.

FIG. 10 is a perspective view of portions of the system of FIG. 8 in use between vertebrae.

FIG. 11 is a perspective view of portions of the system of FIG. 8 in use between vertebrae.

FIG. 12A is a perspective view from a generally anterior direction of portions of the system of FIG. 1.

FIG. 12B is a perspective view from a posterolateral direction of the portions of the system of FIG. 1 illustrated in FIG. 12A.

FIG. 13 is a perspective view from a posterolateral direction of portions of the system of FIG. 1.

FIG. 14 is a perspective view from a generally anterior direction of portions of the system of FIG. 1.

### DETAILED DESCRIPTION

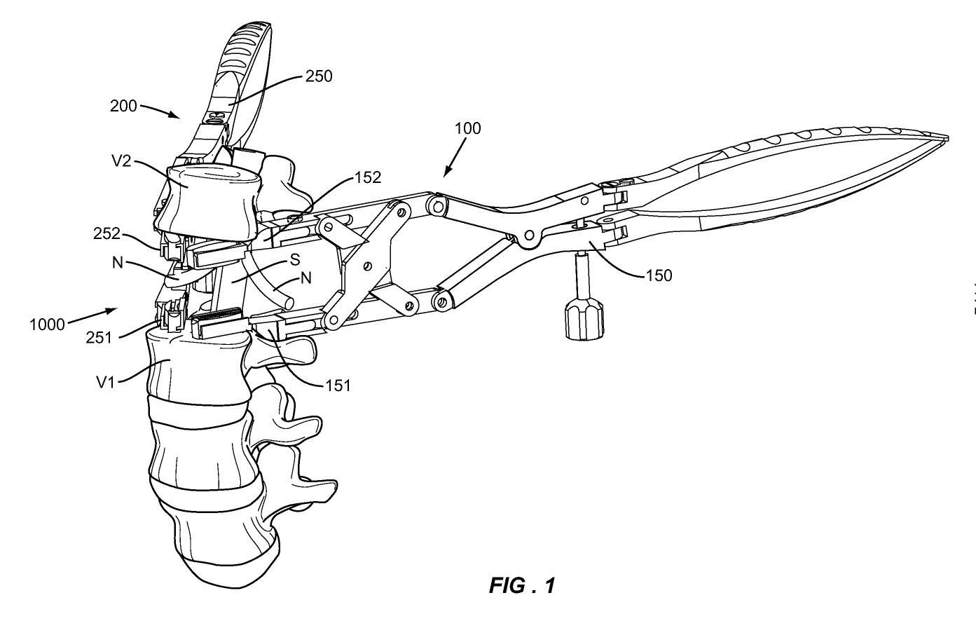

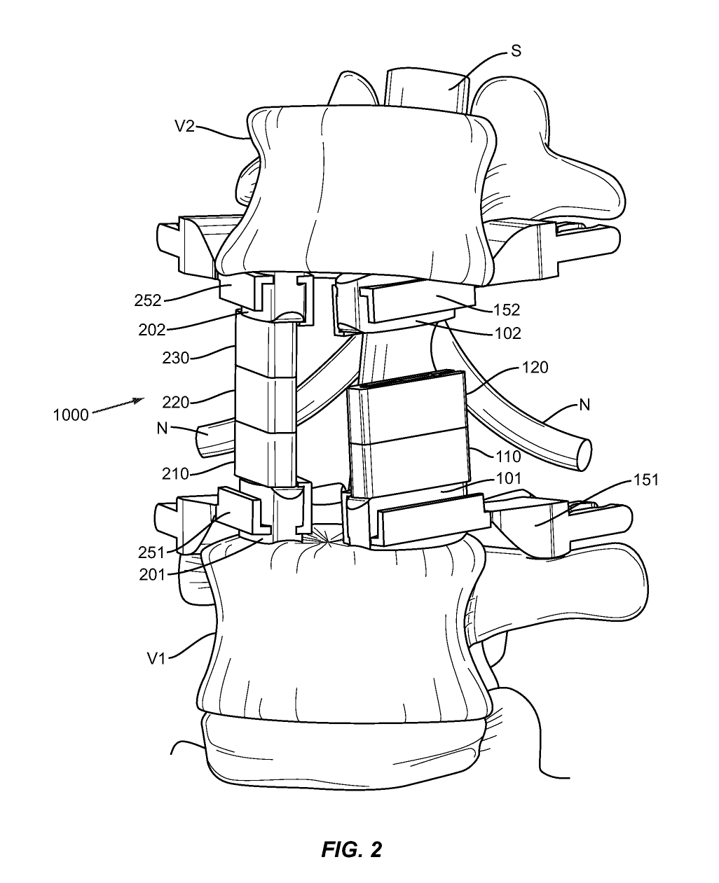

Portions of a vertebral body replacement system 1000 in use between vertebrae V1 and V2 are illustrated in FIGS. 1 and 2. As shown, a vertebra has been removed from between vertebrae V1 and V2. For some embodiments, more than one vertebra may be removed from between vertebra such as V1 and V2 that will be adjacent to a replacement system, or all or part of a vertebra may be left between the vertebrae V1 and V2 during a procedure using a vertebral body replacement system. A spinal cord S and nerve roots N are also illustrated. The embodiment shown may include insertion from bilateral posterolateral approaches as illustrated. The vertebral body replacement system 1000 shown in FIG. 1 includes a primary device 100 and a secondary device 200. In some embodiments, the primary device 100 or the secondary device 200 may be used alone or the devices may be used in combination with one another or with other devices to replace at least part of the function of a vertebral body. For each of the primary device 100 and the secondary device 200 illustrated, a spreader instrument 150, 250 is provided. The spreader instrument 150 includes a first tip 151 and a second tip 152. The illustrated spreader instrument 150 is operable to move the first tip 151 and the second tip 152 farther apart from one another and closer to one another. The spreader instrument 250 includes a first tip 251 and a second tip 252. The illustrated spreader instrument 250 is operable to move the first tip 251 and the second tip 252 farther apart from one another and closer to one another.

In FIG. 1, the spreader instruments 150, 250 are shown in a partially expanded state with components of the devices in contact with the vertebra V1, but not in contact with the vertebra V2. In FIG. 2, the spreader instruments 150, 250 (FIG. 1) have been operated to move the respective first tips 151, 251 farther apart from the second tips 152, 252. Operation of the spreader instruments 150, 250 in this manner may be effective to distract and hold in place the vertebrae V1, V2. The illustrated spreader instruments 150, 250 include an “X” linkage that provides for substantially parallel separating ends of the spreader instruments 150, 250. In other embodiments, a spreader instrument may be configured to include any other operable linkage, gear, lever, or mechanism to affect spreading, and maybe driven by, without limitation, compression, expansion, pushing, pulling, or twisting by an operator, a threaded shaft, an electric motor, or a pneumatic device.

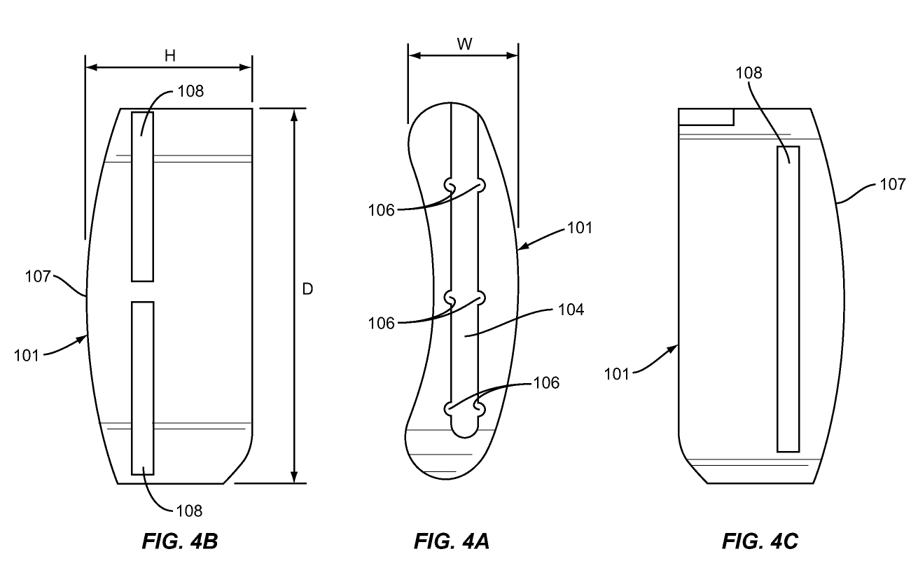

A first end component 101 is shown in FIGS. 2-7. The first end component 101 has a height H, a width W, and a depth D, as illustrated in FIGS. 4A and 4B. General Note: The descriptors “height H,” “width W,” and “depth D” are used throughout this description to specify relative heights, widths, and depths of particular components and are not intended to compare heights, widths, or depths of different components. Here, for example, the height H, width W, and depth D of the first component 101 are described without reference to other components.

The depth D of the illustrated embodiment of the first end component 101 is greater than the width W. As illustrated in FIG. 2, the first end component 101 is configured to couple with the first tip 151. The coupling between the first end component 101 and the first tip 151 may be a sliding coupling, with a portion of the first tip 151 entering into notches 108 on the sides of the first end component 101, as illustrated with a combination of FIGS. 2, 4B, and 4C. In other embodiments, coupling between a first tip and a first end component may be accomplished by any effective mechanism or structure, including without limitation, clamping, a threaded shaft and threaded hole, or an expanding shaft or holding portion. As shown, the extent of the height 107 (FIGS. 4B and 4C) of the first end component 101 is configured to be placed against the vertebra V1 (FIG. 2). A surface of the first end component 101 that is placed against the vertebra V1 may include a treatment to increase friction with the vertebra V1 or to induce bone growth into the first end component 101. For example and without limitation, the surface may include one or more keels, teeth, roughenings, ratchets, spikes, indents, knurling, screws, or any variety of coatings or surfaces to induce bone growth into the first end component 101.

The vertebral body replacement system 1000 may also include a second end component 102 having a height, a width, and a depth. As illustrated in FIGS. 2 and 5-7, the second end component 102 may essentially be of a mirror image shape compared with the first end component 101. In some embodiments, the second end component 102 includes a depth that is greater than its width. As illustrated in FIG. 2, the second end component 102 is configured to couple with the second tip 152. The coupling between the second component 102 and the second tip 152 may be a sliding coupling, with a portion of the second tip 152 entering into notches 109 on the sides of the second component 102, as illustrated with a combination of FIGS. 2 and 5-7. In other embodiments, coupling between a second tip and a second component may be accomplished by any effective mechanism or structure, including without limitation, clamping, a threaded shaft and threaded hole, or an expanding shaft or holding portion. An extent of the height of the second end component 102 is configured to be placed against the vertebra V2, as shown in FIG. 2. A surface of the second end component 201 that is placed against the vertebra V2 may include a treatment to increase friction with the vertebra V2 or to induce bone growth into the second end component 201. For example and without limitation, the surface may include one or more keels, teeth, roughenings, ratchets, spikes, indents, knurling, screws, or any variety of coatings or surfaces to induce bone growth into the second end component 201.

One or both of the end components 101, 102 of the primary device 100 may have a respective width W that is much smaller than its respective depth D so that each end component 101, 102 may be inserted through a narrow opening. For example and without limitation, with regard to one or both of the end components 101, 102 respectively, the depth D may be between about two and twenty times the width W, the depth D may be greater than ten times the width W, or the depth D may be greater than four times the width W.

The vertebral body replacement system 1000 may also include one or more central components configured to be placed between end components. As illustrated in FIGS. 2 and 5-7, the central components for the primary and secondary devices 100, 200 respectively include lower central components 110, 210, middle central components 120, 220, and upper central component 230. An additional upper central component for the primary device 100 may be included in some embodiments, but is not illustrated in FIGS. 2 and 5-7 so that other features may be emphasized. In some embodiments, two or more of the central components may be integrated so that there are two central components or only one central component between the end components. One or more central components may be integrated with an end component. Additionally, in some embodiments, more than three central components may be used between a pair of end components.

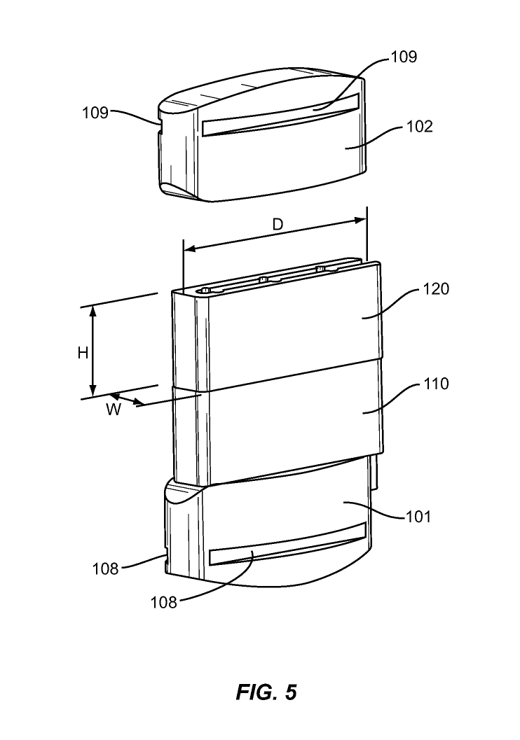

An embodiment of the middle central component 120 is shown in FIG. 5 with a height H, a width W, and a depth D. The depth D is greater that the width W respectively for each of the middle central component 120 and the lower central component 110 shown. In some embodiments, the respective width W is much smaller than the respective depth D so that a central component may be inserted through a narrow opening. For example and without limitation, with regard to one or more of the central components respectively, the depth D may be between about two and twenty times the width W, the depth D may be greater than ten times the width W, or the depth D may be greater than four times the width W.

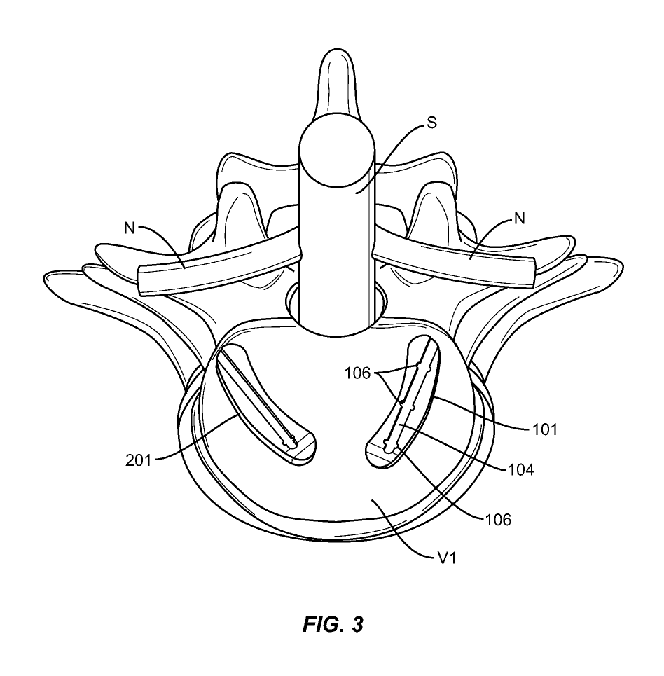

A surface of one or both of the first end component 101 and the second end component 102 that is configured to be directed toward the at least one central component may include an opening configured to receive a portion of the at least one central component. As shown in FIGS. 3, 4A, 6, and 7, an opening 104 with a slot shape in the first end component 101 is configured to receive a tenon 115 of the lower central component 110. In other embodiments, an opening may be of any functional shape to receive a portion of a central component. The opening 104 includes indents 106 configured to receive bumps 116 on the tenon 115. The interface between the indents 106 and the bumps 116 may provide a more secure connection in some embodiments. The bumps 116 may have resilient properties that allow them to be compressed in portions of the opening 104 where there are not indents, and then to expand in the indents 106. Alternatively or in addition, the opening 104 may flex open to allow passage of the bumps 116 through the opening 104. The number of indents and bumps can be altered in various embodiments. Additionally, there may be a different number of indents than bumps on some components that will be coupled together.

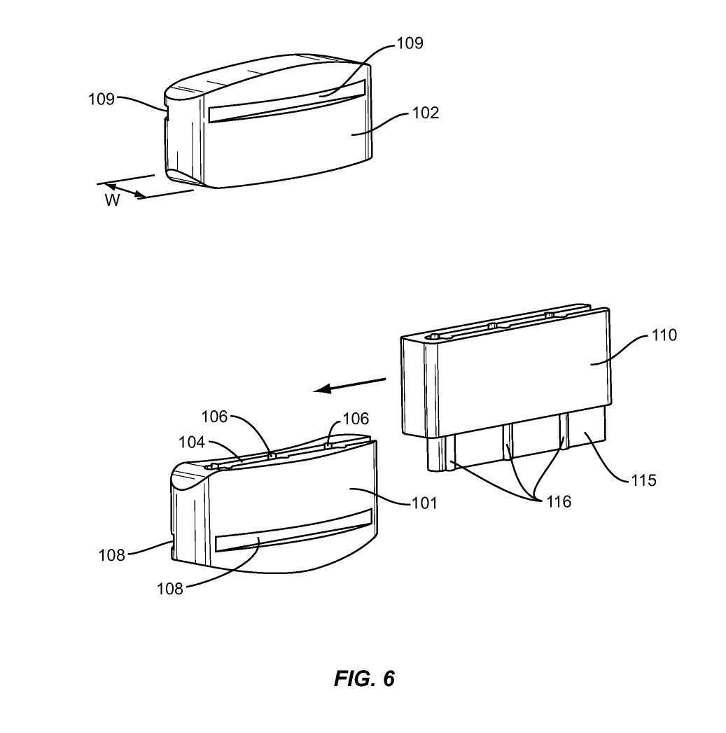

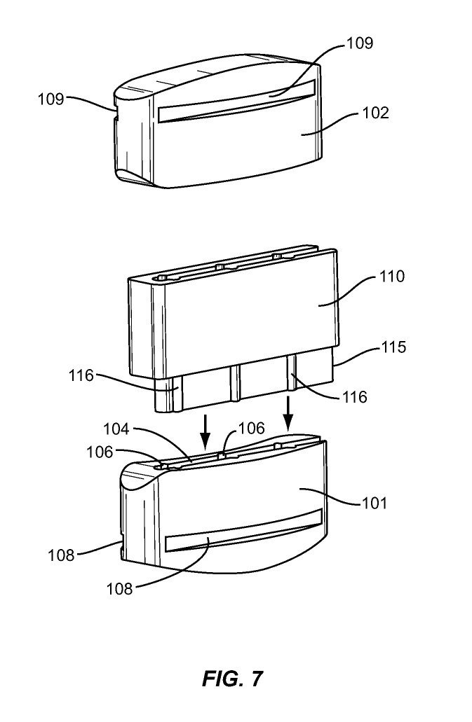

In FIG. 6, the lower central component 110 is configured to slide along the depth of the first end component 101, as depicted by the arrow, into a coupled relationship with the first end component 101. One or all of the tenon 115 and the bumps 116 may be compressed and the opening 104 may be flexed to a wider state in order to slide the tenon 115 through the opening 104 until the bumps 116 seat in the indents 106. In FIG. 7, the lower central component 110 is configured to be pushed transversely into an extent of the height of the first end component 101, as depicted by the arrows, into a coupled relationship with the first end component 101. One or all of the bumps 116 may be aligned with and seated in the indents 106. Alternatively or in addition, any other functional connection between an end component and a central component, including an opening in the central component and a portion extending away from the end component to fit in the opening in the central component, may be used. Various cooperating combinations of central components and end components may be used that include components that have two openings on some components and that have two portions extending away from a component on some components.

Second, third, or greater numbers of central components may be added to the primary device 100 or the secondary device 200 in various embodiments. Central components of some embodiments may be of various heights, and some embodiments may include one or more central components that are of a variable height. Each coupling between components may be similar to the couplings specifically described herein, or may be of any type that is structurally adequate to assemble the vertebral body replacement system 1000.

Elements of the secondary device 200 in some embodiments are similar to elements of the primary device 100, and may be approximately mirror image shaped. Therefore, the descriptions of the primary device 100 herein may be applied to similar elements of the secondary device 200.

As specifically disclosed in FIGS. 1-3, the spreader instrument 250 includes a first tip 251 and a second tip 252. A first end component 201 with a depth greater than its width is configured to couple with the first tip 251. A second end component 202 with a depth greater than its width is configured to couple with the second tip 252. The secondary device 200 includes at least one central component, such as a lower central component 210, a middle central component 220, or an upper central component 230, each with a depth greater than its width. The at least one central component is configured to be placed between the first end component 201 and the second end component 202.

The vertebral body replacement system 1000 may include one or both of the primary device 100 and the secondary device 200, and the devices 100, 200 may be placed at other positions between vertebrae than those depicted herein. Each device 100, 200 may be separately or sequentially operated to cooperatively distract a space, or both may be operated simultaneously.

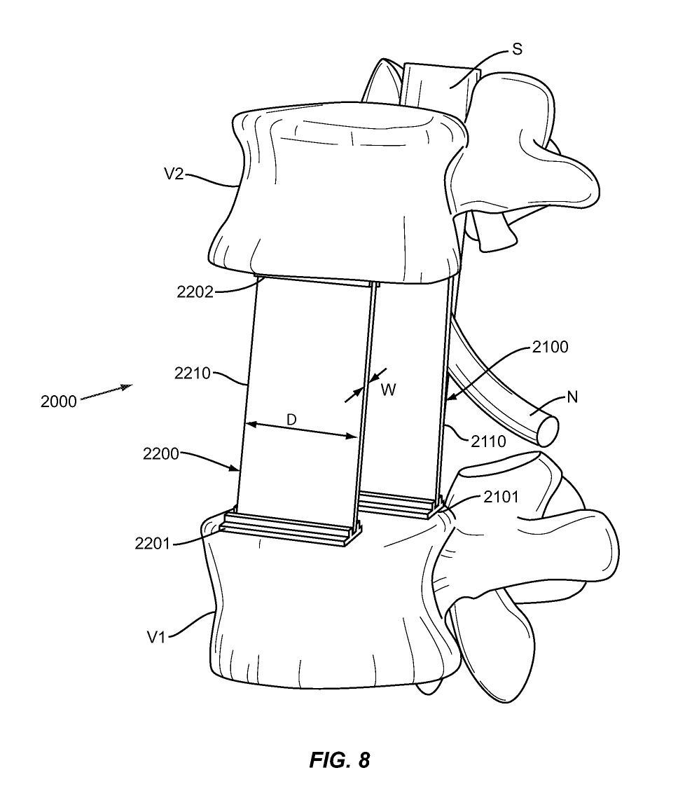

An embodiment of a vertebral body replacement system 2000 is illustrated in FIGS. 8-11. As shown, a vertebra has been removed from between vertebrae V1 and V2. For some embodiments, more than one vertebra may be removed from between vertebra such as V1 and V2 that will be adjacent to the replacement system, or all or part of a vertebra may be left between the vertebrae V1 and V2 during a procedure using the vertebral body replacement system 2000. A spinal cord S and nerve roots N are also illustrated. The system may include insertion from a lateral approach as illustrated. The vertebral body replacement system 2000 shown includes a primary device 2100 and a secondary device 2200. In some embodiments, the primary device 2100 or the secondary device 2200 may be used alone or the devices may be used in combination with one another or with other devices to replace at least part of the function of a vertebral body. For each of the primary device 2100 and the secondary device 2200 illustrated, a spreader instrument may be provided with similar operational capabilities to those detailed herein with regard to the spreader instruments 150, 250 for the primary device 100 and the secondary device 200.

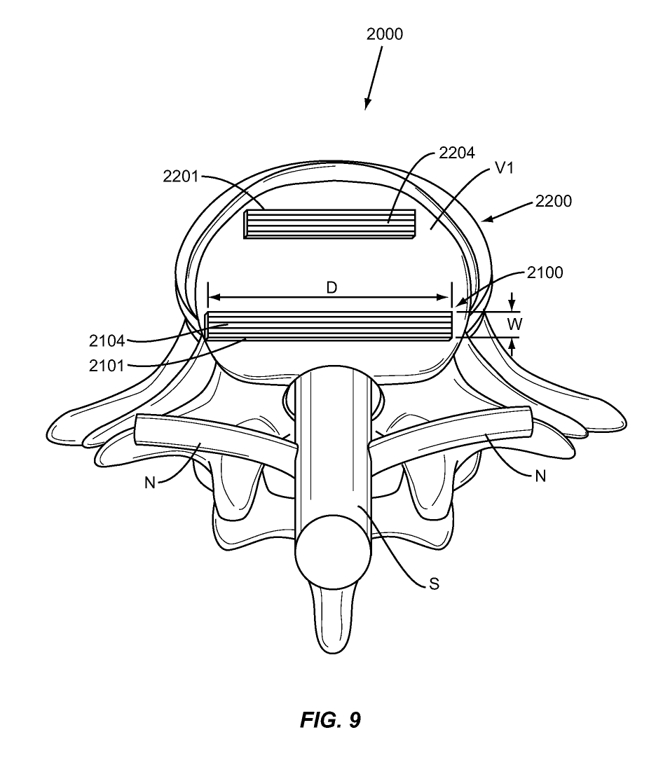

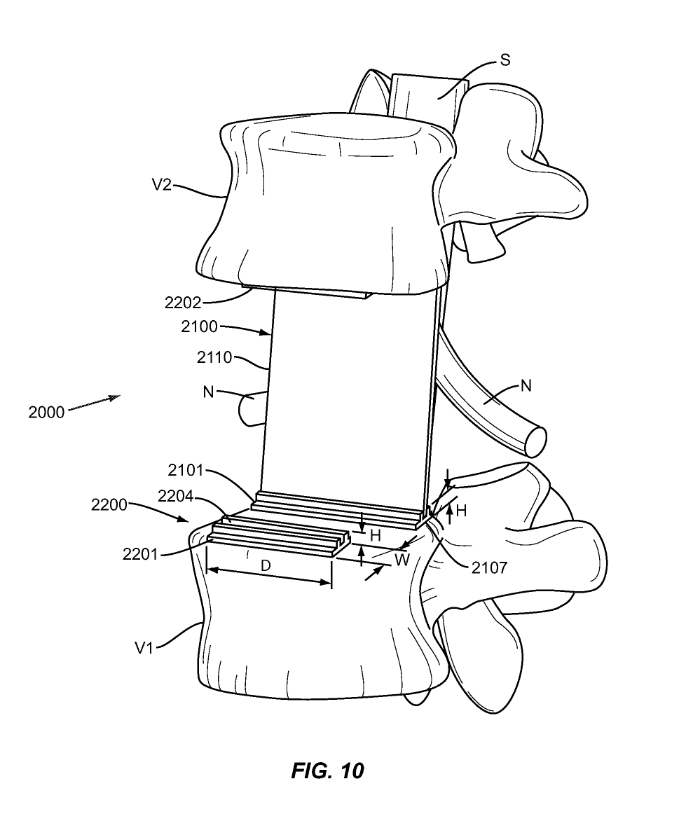

A first end component 2101 is shown in FIGS. 8-11. The first end component 2101 has a height H, a width W, and a depth D, as illustrated in FIGS. 9 and 10. The depth D of the illustrated embodiment is greater than the width W. Coupling between a first tip of a spreader and the first end component may be provided for in some embodiments. The coupling may be accomplished by any effective mechanism or structure, including without limitation, sliding, clamping, a threaded shaft and threaded hole, or an expanding shaft or holding portion. As shown in FIG. 10, an extent of the height 2107 of the first end component 2101 is configured to be placed against the vertebra V1. A surface of the first end component 2101 that is placed against the vertebra V1 may include a treatment to increase friction with the vertebra V1 or to induce bone growth into the first end component 2101. For example and without limitation, the surface may include one or more keels, teeth, roughenings, ratchets, spikes, indents, knurling, screws, or any variety of coatings or surfaces to induce bone growth into the first end component 2101.

The vertebral body replacement system 2000 may also include a second end component (not shown) having a height H, a width W, and a depth D. The second end component may essentially be the same as the first end component 2101 in some embodiments. The second end component may include a depth D that is greater than its width W. Coupling between a second tip of a spreader and the second end component may be provided for in some embodiments. The coupling may be accomplished by any effective mechanism or structure, including without limitation, sliding, clamping, a threaded shaft and threaded hole, or an expanding shaft or holding portion. An extent of the height of the second end component may be configured to be placed against the vertebra V2. A surface of the second end component that is placed against the vertebra V2 may include a treatment to increase friction with the vertebra V2 or to induce bone growth into the second end component. For example and without limitation, the surface may include one or more keels, teeth, roughenings, ratchets, spikes, indents, knurling, screws, or any variety of coatings or surfaces to induce bone growth into the second end component.

One or both of the end components of the primary device 2100 may have a respective width W that is much smaller than its respective depth D so that the respective end component may be inserted through a narrow opening. For example and without limitation, with regard to one or both of the first and second end components of the primary device 2100, the depth D may be between about two and twenty times the width W, the depth D may be greater than ten times the width W, or the depth D may be greater than four times the width W.

The vertebral body replacement system 2000 may also include one or more central components configured to be placed between end components. As illustrated in FIGS. 8, 10, and 11, single central components 2110 and 2210 (FIG. 8) are provided for the primary and secondary devices 2100, 2200 respectively. In some embodiments, two or more central components may be joined together to form a central component between a pair of end components. One or more central components may be integrated with an end component. Additionally, in some embodiments, more than three central components may be used between a pair of end components. In some embodiments, one or more single central components may be provided with a height that is at least as great as any central component anticipated for use with the system. One or both of these central components may then be altered to a desired height intraoperatively as an effective height is determined by a surgeon or other medical staff. The central components may be cut with a specialized cutter, saw, shearing instrument, or by any other device to alter their height. The central components may include break-off zones where the central components may be reduced in height by bending the central components across their height at a break-off zone. A break-off zone may include a reduced volume of material and may include material that has been hardened to make it more capable of being broken at a particular place. A system may also include a set of trial central components that may be inserted between end components to assist in determining appropriate heights for central components. As depicted in FIG. 8, the depth D is greater that the width W for the central component 2210. In some embodiments, the respective width W is much smaller than the respective depth D so that a central component may be inserted through a narrow opening. For example and without limitation, with regard to one or more of the central components, the depth D may be between about two and twenty times the width W, the depth D may be greater than ten times the width W, or the depth D may be greater than four times the width W.

A surface of one or both of the first end component 2101 and the second end component that is configured to be directed toward the at least one central component 2110 may include an opening configured to receive a portion of the at least one central component. As shown in FIG. 9, an opening 2104 with a slot shape in the first end component 2101 is configured to receive a portion of the central component 2110. In other embodiments, an opening may be of any functional shape to receive a portion of a central component.

Embodiments of the central component 2110 may be configured to slide along the depth of the first end component 2101 into a coupled relationship with the first end component 2101. In some embodiments, the central component 2110 may be configured to be pushed transversely into an extent of the height of the first end component 2101 and into a coupled relationship with the first end component 2101. Alternatively or in addition, any other functional connection between an end component and a central component, including an opening in the central component and a portion extending away from the end component to fit in the opening in the central component, may be used.

Second, third, or greater numbers of central components may be added to the primary device 2100 or the secondary device 2200 in various embodiments. Each coupling between components may be similar to the couplings specifically described herein, or may be of any type that is structurally adequate to assemble the vertebral body replacement system 2000.

Elements of the secondary device 2200 in some embodiments are similar to elements of the primary device 2100, and may be approximately the same shape. In the illustrated embodiment of FIGS. 8-11, the secondary device 2200 has a smaller depth than the primary device 2100. Descriptions of the primary device 2100 herein may be applied to similar elements of the secondary device 2200. The secondary device 2200 includes at least one central component, such as the central component 2210 with a depth greater than its width. The at least one central component is configured to be placed between the first end component 2201 and the second end component 2202.

The vertebral body replacement system 2000 may include one or both of the primary device 2100 and the secondary device 2200, and the devices 2100, 2200 may be place at other positions between vertebrae than those depicted herein. Each device 2100, 2200 may be separately or sequentially operated to cooperatively distract a space, or both may be operated simultaneously. Central components of varying heights or with wedge shapes may be used in the devices 2100, 2200 to achieve sagittal and coronal angulation between the vertebrae V1, V2. In some embodiments, these angulations may be used to restore proper curvature of a spine in any plane or combination of planes.

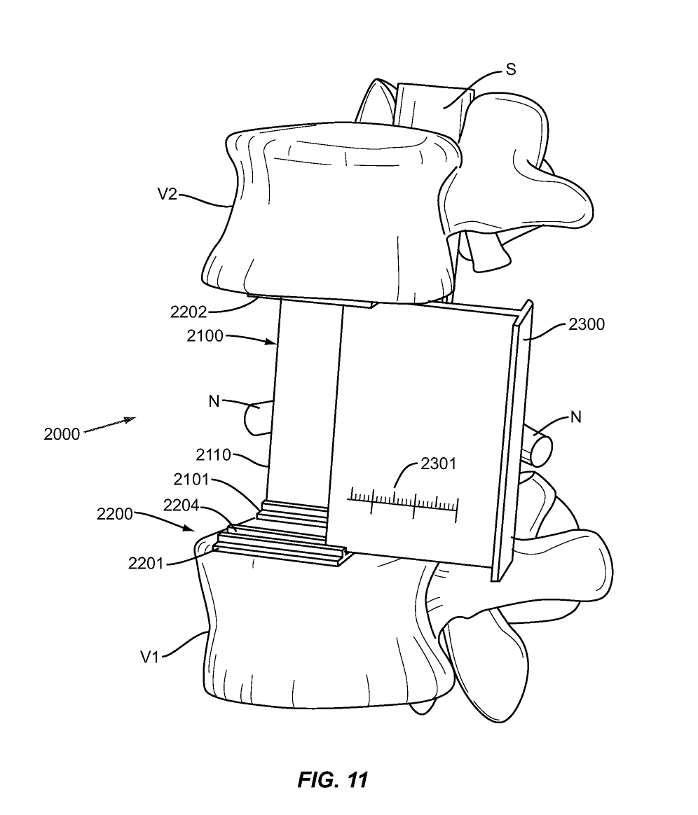

As shown in FIG. 11, the first end component 2201 with the opening 2204 and a similar opening in the second end component 2202 are configured to receive an osteotome 2300 that may move or slide along the tracks or openings. The illustrated osteotome 2300 slides in openings in the first and second end components 2201, 2202 to cut tissue between the first and second end components 2201, 2202. The osteotome 2300 is straight between the first and second end components 2201, 2202, but in other embodiments may be curved or specially configured to avoid anatomical structures. The illustrated osteotome 2300 includes depth markings 2301 to provide an indication of the depth to which the osteotome 2300 has been driven into an anatomical structure. Alternatively or in addition, an osteotome may include positive stops relative to one or both of the first and second end components 2201, 2202, or relative to an access instrument through which an osteotome is introduced, or relative to any other instrument or implant used in a related procedure. A second osteotome may be used in some embodiments between end components of the primary device 2100. Alternatively, the osteotome 2300 may separately be employed in a similar way between the end components of the primary device 2100.

A tissue removal instrument or instrument set may be used in some embodiments to remove tissue relative to or adjacent to the osteotome 2300. Any cutting, reaming, milling, or chiseling instrument or instrument set may be used in various embodiments. The following U.S. Pat. Nos. include disclosure describing various cutting, reaming, milling, and chiseling instruments or instrument sets that may be used to remove tissue relative to or adjacent to the osteotome 2300: U.S. Pat. Nos. 5,741,253; 6,083,228; 6,159,214; 6,224,607; 6,440,139; 6,517,544; 6,537,279; 6,692,501; 6,966,912; 6,986,772; 7,083,623; 7,160,304; and 7,211,085. Each of these patents is hereby incorporated by reference in its entirety to the present disclosure. While the incorporated disclosure is primarily directed to removing material from disc spaces, the same instruments and techniques with altered proportions are applicable to removal of all or part of vertebrae. Any other instrument or instrument set that is capable of removing tissue from between or adjacent to the central components 2110, 2210 is contemplated.

Any embodiment of the vertebral body replacement system may be used in conjunction with a fill material. Fill material may be placed in or around any of the components of the vertebral body replacement system. For example, fill material may be placed adjacent to or fully or partially encapsulating or filling any of the components of the vertebral body replacement system. In addition, fill material may be placed in a membrane or bag to contain the fill material in a desired location in or near a component of the vertebral body replacement system. The fill material may be a fluid, and then harden or cure in place. The fill material may be a paste, gel, liquid, suspension, granular mixture, or similar substance. Non-limiting examples of fill materials include bone cement, paste, morselized allograft, autograft, or xenograft bone, ceramics, or various polymers. An example bone cement is polymethylmethacrylate (PMMA), which may be made from methylmethacrylate, polymethylmethacrylate, esters of methacrylic acid, or copolymers containing polymethylmethacrylate and polystyrene. Additional non-limiting examples of the fill material include semi-rigid flowable or hardenable material such as silicone or various types of urethane materials. It should further be understood that other types of fill materials which are not necessarily hardenable or curable may be used. For example, the fill material may comprise beads or small particles or grains of material, some of which may, in aggregate, achieve a harder consistency as a result of interlocking or compaction. In some embodiments, the fill material may also include a bone growth promoting substance.

Fill material may be used unconstrained, or fill material may be injected into a membrane or bag. The membrane or bag may be constructed, in whole or in part, of a non-permeable material. The membrane or bag may include compliant or non-compliant balloon materials such as those commonly used to manufacture coronary and Kyphoplasty medical devices. Such materials may include, but not be limited to, mylar, rubber, polyurethane, vinyl, latex, polyethylenes, ionomer, and polytetrapthalate (PET), as well as less flexible materials such as Kevlar®, PEBAX®, stainless steel, titanium, nickel-titanium alloys, and other metals and alloys and/or ceramics. A compliant membrane or bag may include reinforcing to limit one or both of the size and shape of the membrane to a clinically advantageous extent. A non-compliant membrane or bag may expand more elastically to more completely fill an irregular opening, depending on the amount of material introduced into the membrane or bag. One or both of the membrane or the bag may be constructed, in whole or in part, of a permeable material, which allows a certain amount of fill material to pass through the membrane or bag. All or a portion may be made permeable by fabricating a material, including but not limited to the membrane materials listed above, into a fabric, weave, mesh, composite, bonded fiber assembly, or any other manufacture known to those skilled in the art.

For embodiments of the system disclosed herein, implanted components and the size or shape of the membrane or bag may be limited to only fill a particular portion of a vertebral space. For example, and without limitation, implant components and a membrane or bag may be configured to only occupy a lateral portion of a vertebral space to accomplish a hemi-vertebrectomy.

Embodiments of the system may include components that in whole or in part are constructed of biocompatible materials of various types. Examples of component materials include, in whole or in part, biocompatible materials of various types. Non-limiting examples of component materials include titanium, titanium alloys, cobalt chrome alloys, stainless steel, ceramics, various plastics, plastic composites, non-reinforced polymers, reinforced polymers, carbon-reinforced polymer composites, PEEK, PEAK, and PEEK composites, and combinations thereof. If any trial instrument or implant is made from radiolucent material, radiographic markers can be located on the trial instrument or implant to provide the ability to monitor and determine radiographically or fluoroscopically the location of the implant. In some embodiments, the implant or individual components of the implant may be constructed of solid sections of bone or other tissues. Tissue materials include, but are not limited to, synthetic or natural autograft, allograft, or xenograft, and may be resorbable or non-resorbable in nature. Examples of resorbable materials that may be used include, but are not limited to, polylactide, polyglycolide, tyrosine-derived polycarbonate, polyanhydride, polyorthoester, polyphosphazene, calcium phosphate, hydroxyapatite, bioactive glass, and combinations thereof.

Embodiments of the invention may be applied to one or all of the lumbar spinal region, the cervical spinal region, and the thoracic spinal region, or between other skeletal structures. Some embodiments may also include supplemental fixation devices in addition to or as part of the vertebral body replacement system for further stabilizing the anatomy. For example, and without limitation, rod and screw fixation systems, anterior, posterior, or lateral plating systems, facet stabilization systems, spinal process stabilization systems, and any devices that supplement stabilization may be used as a part of or in combination with embodiments of the vertebral body replacement system.

Embodiments of the system may be implanted, for example, from a generally posterolateral approach or a lateral approach. However, embodiments of the invention may include implantation of one or more components from any surgical approach, including but not limited to, posterior, anterior, anterolateral, transpedicular, lateral extracavitary, in conjunction with a laminectomy, in conjunction with a costotransversectomy, or by any combination of these and other approaches.

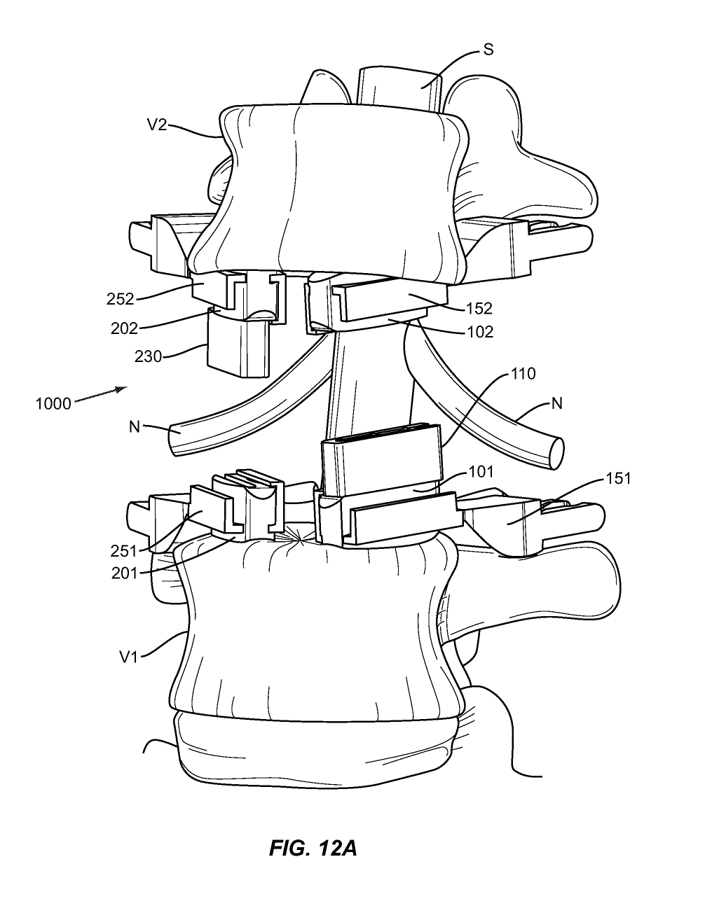

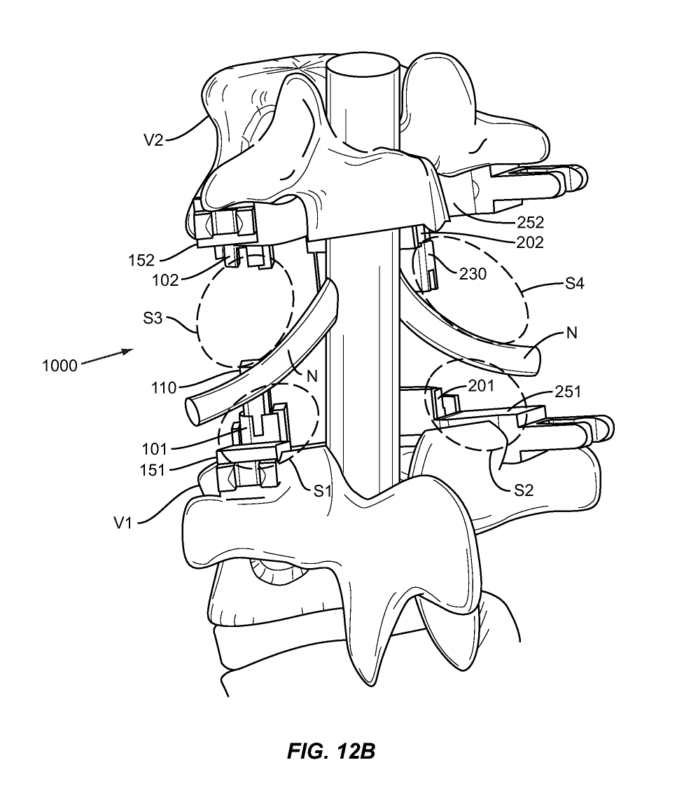

A method embodiment of stabilizing a spine from which a vertebral body will be at least partially removed includes placing a first end component against a first adjacent vertebral body, including moving the first end component through a space between the first adjacent vertebral body and a nerve root extending from a spinal cord. For example, as shown in FIGS. 12A and 12B, both first end components 101, 201 have been placed against adjacent vertebral body V1. The first end components 101, 201 have been respectively moved through spaces 51, S2 (FIG. 12B) between the adjacent vertebral body V1 and the nerve roots N that extend from the spinal cord S. In the illustrated embodiment, the first end components 101, 201 have been respectively coupled to the first tips 151, 251 that are part of the spreader instruments 150, 250 (FIG. 1).

Method embodiments may include placing a second end component against a second adjacent vertebral body opposite from the first end component. For example, one or both of second end components 102, 202 may be placed against the adjacent vertebral body V2. Either or both of the second end components 102, 202 may be placed directly against the adjacent vertebral body V2 upon insertion, or may be moved into place at some distance from the adjacent vertebral body V2 and later moved against the adjacent vertebral body V2 by expansion of the spreader instrument 150, 250 (FIG. 1), or by another act. In the illustrated embodiment, the second end components 102, 202 have been respectively coupled to the second tips 152, 252 that are part of the spreader instruments 150, 250. One or both of the spreader instruments 150, 250 may be moved into position in a spinal column as shown in FIG. 1 and then operated to move the first end components 101, 201 farther apart from the respective second end components 102, 202, as illustrated in FIGS. 12A, 12B, 13, and 14.

Some method embodiments may include inserting a first central component between the first end component and the second end component, including moving the first central component through a space between the second end component and the nerve root extending from the spinal cord. Two examples of such an insertion are illustrated in FIGS. 12A and 12B. In a first example, the first central component inserted is the lower central component 110 that is placed between the first end component 101 and the second end component 102 by moving the lower central component 110 through a space S3. The space S3 is between the second end component 102 and the nerve root N. In this example, the lower central component 110 may be moved transversely into an extent of the height of the first end component 101, as is described in greater detail herein in association with FIG. 7. In a second example, the first central component inserted is the upper central component 230 that is placed between the first end component 201 and the second end component 202 by moving the upper central component 230 through a space S4. The space S4 is between the second end component 202 and the nerve root N. As used herein, descriptions of insertions “between” components do not necessarily mean that the component being inserted is being placed against either referenced component. In some embodiments, such an insertion may include the act of connecting to both referenced components by sliding, transverse attachment, or any other connecting act.

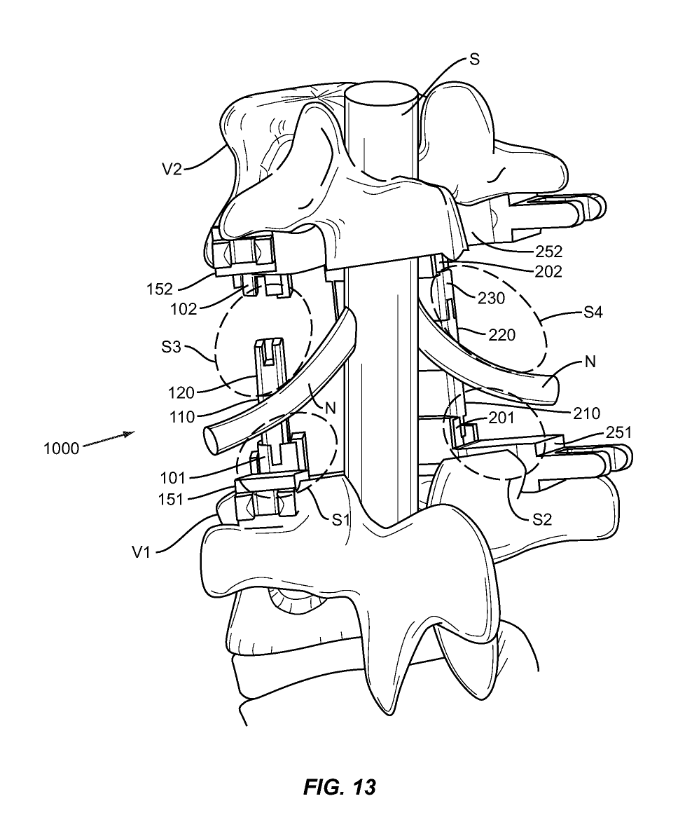

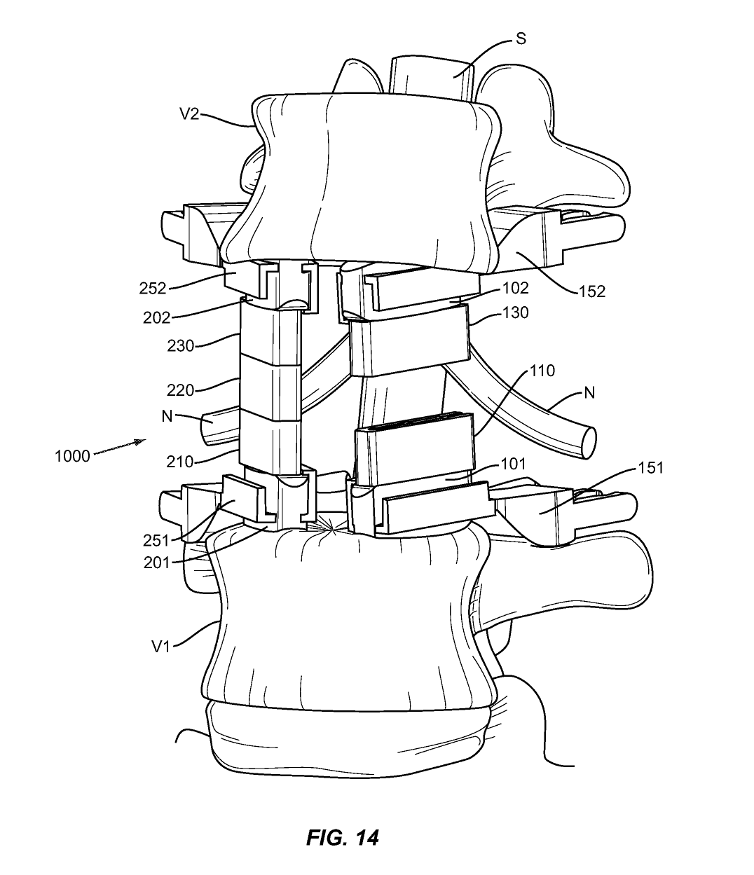

Some method embodiments may include inserting a second central component between the first central component and the second end component, including moving the second central component through a space between the second end component and the nerve root. As applied to the first example of FIGS. 12A and 12B, and as further depicted in FIGS. 13 and 14, the second central component may be either of the middle central component 120 (FIG. 13) or an upper central component 130 (FIG. 14). Either of the middle central component 120 or the upper central component 130 may be moved through the space S3 between the second end component 102 and the nerve root N.

Some method embodiments of stabilizing a spine may include inserting a second central component between the first central component and the first end component, including moving the second central component through the space between the second end component and the nerve root. As applied to the second example of FIGS. 12A and 12B, and as further depicted in FIGS. 13 and 14, the second central component may be either of the lower central component 210 or the middle central component 220 that may be inserted after the upper central component 230. Either of the lower central component 210 or the middle central component 220 may be moved through the space S4 between the second end component 202 and the nerve root N. The lower central component 210 may be inserted through the space S4, over the nerve root N, and onto the first end component 201, similar to the insertion depicted in FIG. 7.

Some method embodiments may include inserting a second central component between the first central component and the first end component, including moving the second central component through the space between the first end component and the nerve root. As applied to the second example of FIGS. 12A and 12B, and as further depicted in FIGS. 13 and 14, the second central component may be either of the lower central component 210 or the middle central component 220 that may be inserted after the upper central component 230. Either of the middle central component 220 or the upper central component 230 may be moved through the space S2 between the first end component 201 and the nerve root N. The middle central component 220 may be inserted through the space S2 and then lifted behind the nerve root N, and coupled with the upper central component 230. The lower central component 210 may be inserted through the space S2 to couple with the first end component 201, similar to the insertion depicted in FIG. 6.

The spaces 51, S2, S3, and S4 specified near various nerve roots may be reasonably altered in size and shape by intraoperatively mobilizing one or more of the nerve roots. Therefore, depictions of the spaces 51, S2, S3, and S4 shown herein should be considered as representative, but are not necessarily precise in size or shape.

In some embodiments, after one or both of the devices 100, 200 is constructed to provide support between vertebrae V1 and V2, spreader instruments 150, 250, along with their respective tips 151, 152, 251, 252, are removed from a patient by sliding the spreader instruments away from a patient in a direction opposite from the direction of insertion. Because the tips 151, 152, 251, 252 may be offset from substantial contact with the vertebrae V1, V2, as they are shown to be in FIGS. 1, 2, and 12A-15, the spreader instruments 150, 250 may be removed from a patient without significantly contacting and potentially damaging the vertebrae V1, V2.

A method embodiment may include performing a vertebral body replacement by implanting a first end component that includes a first track against a first adjacent vertebral body. The first adjacent vertebral body is on a first side of the vertebral body to be replaced, and the first track is oriented toward the vertebral body to be replaced. The method may also include implanting a second end component that includes a second track against a second adjacent vertebral body. The second vertebral body is on a second side of the vertebral body to be replaced that is opposite from the first side, and the second track is oriented toward the vertebral body to be replaced. By way of example, the first end component 2201 of the secondary device 2200 shown in FIGS. 8-11 includes the opening 2204 with a slot shape that may serve as a first track. The first end component 2201 is implanted against the first vertebral body V1. The second end component 2202 shown in FIGS. 8, 10, and 11 is in this embodiment the same size and shape as the first end component 2201, but is implanted against the second vertebral body V2 so that it has a second track that is oriented downwardly, as drawn, toward the vertebral body to be replaced. As illustrated, this method is accomplished from a lateral approach to a spine, but may be accomplished from other approaches.

As illustrated in FIG. 11, the osteotome 2300 may be moved in the first track and the second track to cut tissue between the first track and the second track. Some embodiments may include another set of tracks that include third and fourth end components and associated tracks, as illustrated by the openings and tracks of the primary device 2100 illustrated in FIGS. 8-11. A second osteotome may be moved in these third and fourth tracks of the primary device 2100 to cut tissue between the third and fourth tracks.

A tissue removal instrument or instrument set that includes various cutting, reaming, milling, and chiseling devices, as disclosed herein, may be used to remove tissue from between two or more of the first, second, third, and fourth tracks. Tissue removal instruments or instrument sets may be guided along any or all of the tracks or any devices that span between the tracks. In some embodiments, one or more osteotomes or a tissue removal instrument may be incorporated into a spreader instrument used to distract or hold distraction between vertebrae V1, V2. In some embodiments, a tissue removal instrument or instrument set may be used to remove substantially all of a vertebral body and other tissues from between two or more of the tracks. In other embodiments, a majority of tissue may be removed with other instruments and methods, and a tissue removal instrument or instrument set may be used to prepare specific clearance for supporting components of a vertebral body replacement system or to prepare vertebrae for bone ingrowth or bone growth.

Various method embodiments are described herein with reference to particular vertebral body replacement systems. However, in some circumstances, each disclosed method embodiment may be applicable to each of the vertebral body replacement systems, or to some other system operable as disclosed with regard to the various method embodiments.

Terms such as lower, upper, middle, center, end, downward, anterior, posterior, adjacent, and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A vertebral body replacement system comprising:
a spreader instrument with a first tip and a second tip, wherein the spreader instrument is operable to move the first tip and the second tip farther apart from one another and closer to one another;
a first end component having a height, a width, and a depth, wherein the depth is greater than the width, and wherein the first end component is configured to couple with the first tip, and wherein an extent of the height of the first end component is configured to be placed against a first adjacent vertebral body;
a second end component having a height, a width, and a depth, wherein the depth is greater than the width, and wherein the second end component is configured to couple with the second tip, and wherein an extent of the height of the second end component is configured to be placed against a second adjacent vertebral body; and
at least one central component having a height, a width, and a depth, wherein the depth is greater than the width, and wherein the at least one central component is configured to be placed between the first end component and the second end component.

2. The system of claim 1 wherein the depth of the first end component is greater than four times the width of the first end component, and wherein the depth of the second end component is greater than four times the width of the second end component.

3. The system of claim 1 wherein a surface of one or both of the first end component and the second end component that is configured to be directed toward the at least one central component includes an opening configured to receive a portion of the at least one central component.

4. The system of claim 1 wherein the depth of the at least one central component is greater than four times the width of the at least one central component.

5. The system of claim 1 wherein the first central component is configured to slide along the depth of the first end component into a coupled relationship with the first end component.

6. The system of claim 1 wherein the first central component is configured to be pushed transversely into an extent of the height of the first end component into a coupled relationship with the first end component.

7. The system of claim 1, further comprising a second central component configured to couple with the at least one central component between the first end component and the second end component.

8. The system of claim 1, further comprising a fill material to be placed between the first adjacent vertebral body and the second adjacent vertebral body.

9. The system of claim 1 wherein the first end component includes a first track along at least a portion of the depth of the first end component and the second end component includes a second track along at least a portion of the depth of the second end component, and further comprising a first osteotome configured to move in one or both of the first track and the second track to cut tissue between the first track and the second track.

10. The system of claim 9, further comprising a second osteotome configured to cut tissue adjacent to the first osteotome.

11. The system of claim 9, further comprising a tissue removal instrument set configured to remove tissue adjacent to the first osteotome.

12. The system of claim 1, further comprising a secondary: spreader, first end component, second end component, and at least one central component; all to be implanted juxtaposition the spreader, first end component, second end component and at least one central component.

13. A method of stabilizing a spine from which a vertebral body will be at least partially removed comprising:
placing a first end component against a first adjacent vertebral body, including moving the first end component through a space between the first adjacent vertebral body and a nerve root extending from a spinal cord;
placing a second end component against a second adjacent vertebral body opposite from the first end component; and
inserting a first central component between the first end component and the second end component, including moving the first central component through a space between the second end component and the nerve root extending from the spinal cord.

14. The method of claim 13, further comprising coupling the first end component and the second end component to a spreader instrument prior to placing the first end component and the second end component and operating the spreader instrument to move the first end component farther apart from the second end component.

15. The method of claim 13, further comprising inserting a second central component between the first central component and the second end component, including moving the second central component through the space between the second end component and the nerve root.

16. The method of claim 13, further comprising inserting a second central component between the first central component and the first end component, including moving the second central component through the space between the second end component and the nerve root.

17. The method of claim 13, further comprising inserting a second central component between the first central component and the first end component, including moving the second central component through the space between the first end component and the nerve root.

18. A method of performing a vertebral body replacement comprising:
implanting a first end component that includes a first track against a first adjacent vertebral body, wherein the first adjacent vertebral body is on a first side of the vertebral body to be replaced, and wherein the first track is oriented toward the vertebral body to be replaced;
implanting a second end component that includes a second track against a second adjacent vertebral body, wherein the second adjacent vertebral body is on a second side of the vertebral body to be replaced that is opposite from the first side, and wherein the second track is oriented toward the vertebral body to be replaced; and
moving a first osteotome in the first track and the second track to cut tissue between the first track and the second track.

19. The method of claim 18, further comprising operating a tissue removal instrument to remove tissue adjacent to the first osteotome.

20. The method of claim 18, further comprising:
implanting a third end component that includes a third track against the first adjacent vertebral body, wherein the third track is oriented toward the vertebral body to be replaced;
implanting a fourth end component that includes a fourth track against the second adjacent vertebral body, wherein the fourth track is oriented toward the vertebral body to be replaced; and
moving a second osteotome in the third track and the fourth track to cut tissue between the third track and the fourth track.

21. The method of claim 20, further comprising operating a tissue removal instrument to remove tissue from between the first track, the second track, the third track, and the fourth track.
